(19) United States
(12) Patent Application Publication
(10) Pub. No.: US 2010/0174239 A1
(43) Pub. Date: Jul. 8, 2010

(54) Tubing for Fluid Delivery Device

(76) Inventors: Ofer Yodfat, Maccabim-Reut (IL); Doron Bushi, Holon (IL); Gil Senesh, Haifa (IL)

Correspondence Address: MINTZ LEVIN COHN FERRIS GLOVSKY & POPEO, ONE FINANCIAL CENTER, BOSTON, MA, 02111, US

(21) Appl. No.: 12/666,417

(22) PCT Filed: Jun. 25, 2008
(86) PCT No.: PCT/IL08/00864

§ 371 Date: Dec. 23, 2009

Related U.S. Application Data
(60) Provisional application No. 60/937,577, filed on Jun. 27, 2007.

(51) Int. Cl. A61M 5/142

(52) U.S. Cl. 604/153

(57) ABSTRACT

Delivery tubes having a flexible portion and pump mechanisms (4) for use therewith include features that reduce losses of an active therapeutic compound within a fluid passing through the delivery tube (17). The delivery tube can include inner (30) and outer (31) layers that are inert to reaction with the therapeutic compound and that restrict diffusion of other compounds, respectively. Rollers (83) of a peristaltic pump mechanism (80) can include structural features that reduce physical impacts on molecules of the therapeutic compound. Related systems, apparatus, and/or articles are also described.

### CROSS-REFERENCE TO RELATED APPLICATION

This application claims the benefit of U.S. provisional patent application Ser. No. 60/937,577, filed on Jun. 27, 2007 and entitled “Tubing for Fluid Delivery Device” which is incorporated by reference herein in its entirety.

### FIELD

The subject matter described herein relates to delivering fluids subcutaneously into the body. Various aspects of the disclosed subject matter deal with tubing for conveying fluids and with pumping mechanisms for moving fluids within tubing.

### BACKGROUND

Diabetes mellitus is a disease of major global importance that has increased in frequency at almost epidemic rates. The worldwide prevalence in 2006 is 170 million people and predicted to at least double over the next 10-15 years. Diabetes is characterized by a chronically raised blood glucose concentration (hyperglycemia), due to a relative or absolute lack of the pancreatic hormone, insulin. Within the healthy pancreas, beta cells, located in the islets of Langerhans, continuously produce and secrete insulin according to the blood glucose levels and thus maintain near constant glucose levels in the body.

Diabetes mellitus patients require the administration of varying amounts of insulin throughout the day to control their blood glucose levels. In recent years, ambulatory portable insulin infusion devices have emerged as a superior alternative to multiple daily injections of insulin. These devices, which can typically deliver insulin at a continuous basal rate as well as in bolus volumes, were developed to liberate patients from repeated self-administered injections and allow greater flexibility in dose administration. Both basal and bolus volumes are desirably delivered in precise doses, according to individual prescription, because an under or overdose of insulin could be fatal. Therefore, high accuracy and reliability are desirable features for insulin injection devices. Additional features that prevent delivery of unintentional insulin excesses or shortages are also desirable.

Several ambulatory insulin infusion devices are currently available on the market. Devices fitted with disposable syringe-type reservoirs and tubes are described in U.S. Pat. No. 3,631,847 to Hobbs, U.S. Pat. No. 3,771,694 to Kaminski, U.S. Pat. No. 4,657,486 to Julius, and U.S. Pat. No. 4,544,369 to Skakoon. The main drawbacks of these devices are their large size and the weight, caused by the spatial configuration and the relatively large driving mechanism of the syringe and the piston. The relatively bulky device is typically carried in a patient's pocket or attached to the belt or some other article of clothing. Consequently, the fluid delivery tube is typically quite long, often longer than 60 cm, to permit needle insertion in remote sites of the body. Such uncomfortable, bulky devices with long tubes tend to be rejected by the majority of diabetic insulin users, because they tend to disturb regular activities, such as for example sleeping and swimming. Furthermore, negative body image effects can be substantial, especially for younger users such as teenagers. In addition, the presence of the delivery tube excludes some otherwise potentially preferable remote insertion sites, like the buttocks and the extremities.

Fluid deliver devices can include a housing having a bottom surface adapted for contact with the patient's skin, a reservoir disposed within the housing, and an injection needle adapted for communication with the reservoir. These skin adhered devices should be disposed of every 2-3 days like current pump infusion sets. This paradigm was described by Burton in U.S. Pat. No. 5,957,895, Connelly, in U.S. Pat. No. 6,589,229, and by Flaherty in U.S. Pat. No. 6,740,059. Other configuration of the skin adhered pumps are disclosed in U.S. Pat. Nos. 6,723,072 and 6,485,461. The pump can include a single piece that adheres to the patient skin for the entire usage duration. The needle emerges from the bottom surface of the pump and is fixed to the device housing. These so-called “second-generation” skin adhered devices tend to be expensive, bulky and heavy, in some examples because of the use of a reservoir configured as a tubular syringe with a plunger. A further disadvantage can arise because of the need for the entire device, including expensive electronics and driving mechanism, to be disposed of approximately every 2-3 days.

Volume and cost limitations characteristic of previous generations of fluid delivery devices can be mitigated using recently developed, skin-adherable devices, such as for example those described in co-pending and co-owned applications for U.S. patent Ser. Nos. 11/397,115 and 12/116,546 and international application No. PCT/IL06/001276, all of which are incorporated herein by reference in their entireties. In some examples, a fluid delivery device can include at least one disposable part and at least one reusable part. The reusable part can include electronic components and at least part of a pumping mechanism. The disposable part can include a reservoir or reservoirs for the therapeutic fluid or fluids, a short delivery tube, and an exit port. Such fluid delivery devices can also include a remote control unit that allows data acquisition, programming, and user inputs. Assembly of the reusable and disposable parts provides a very thin dimensioned device that is inexpensive, unobtrusive, relatively light, and discreet.

In some examples, the dispensing mechanism of these newer adhered devices can be based on a peristaltic positive displacement pumping device. Insulin or an other therapeutic fluid or fluids can be stored in a bladder type reservoir, and a tube, hereinafter the “delivery tube,” maintains fluid communication between the reservoir and an exit port. This dispensing mechanism can include a rotating structure, such as a wheel and one or more rollers. The rollers consecutively squeeze the delivery tube and displace fluid in the direction of motion. The type of peristaltic mechanism used can be independent of the reservoir shape and size. Accordingly, the reservoir can be symmetrical or unsymmetrical with flexible walls (for example a bladder type reservoir) or rigid or semi-rigid. An unsymmetrical, bladder-type reservoir can be beneficial in thin, skin-adhered fluid dispensing devices. The peristaltic mechanism can also be tailored to a two or more part device such as is described in PCT/IL06/001276. For example, the rotating wheel can be included in a reusable part and the delivery tube in a disposable part. The rotating wheel squeezes the delivery tube only upon operative coupling of the parts thus avoiding creeping of the plastic delivery tube during shelf life.

### SUMMARY

In one aspect, an article includes a delivery tubing for delivering a therapeutic fluid from a reservoir to a patient's body. The therapeutic fluid includes at least one biologically active compound and at least one preservative compound. The delivery tubing has a flexible portion that includes an inner layer that is substantially inert to reactions with the at least one biologically active compound and an outer layer that is substantially impermeable to both liquid and vapor phases of at least the preservative compound and/or water.

In an interrelated aspect, a method includes providing a therapeutic fluid from a fluid reservoir via a delivery tubing. The therapeutic fluid includes at least one biologically active compound and at least one preservative compound. The delivery tubing has a flexible portion comprising an inner layer that is substantially inert to reactions with the at least one biologically active compound and an outer layer that is substantially impermeable to gas and liquid phases of at least the preservative compound and/or water. The method also includes operating a pump motor to rotate a wheel in a direction of rotation. The wheel includes one or more rollers and is disposed such that the one or more rollers squeezes the flexible portion of the delivery tube against a stator to propel the therapeutic fluid in the delivery tubing in the direction of rotation as the wheel rotates. The method also includes delivering the therapeutic fluid to a patient's body via the delivery tubing.

In some variations one or more of the following can optionally be included. The article can optionally also include a pump motor, a stator, and a wheel that is rotated in a direction of rotation by the pump motor. The wheel can optionally include one or more rollers. The wheel can be disposed such that the one or more rollers squeezes the flexible portion of the delivery tube against the stator to propel fluid in the delivery tubing in the direction of rotation as the wheel rotates.

The therapeutic fluid can optionally be insulin. The at least one preservative compound can optionally be one or more of: phenol, phenol derivative, and cresol. The flexible portion of the tubing can optionally include an inner layer and an outer layer, with the inner layer further including a first material that is substantially more inert to insulin than the outer layer which can include an elastomer. The first material can optionally include fluorosilicone.

The rollers can optionally have a convex cross section in a plane parallel to the axis of rotation of the wheel. The stator can optionally include a contact surface that is substantially parallel to the axis of rotation. The flexible portion of the delivery tube can optionally be compressed between the convex cross section of the roller and the stator to form two parallel lumens within the delivery tubing. The stator can optionally include a concave cross section. A valve can be include to allow unidirectional propulsion of the therapeutic fluid in the delivery tubing.

Various optional configurations for the delivery tubing are possible. It can optionally further include a rigid portion; the rigid portion of the delivery tube is substantially impermeable to at least one of the therapeutic fluid components and/or to CO₂ than the flexible portions of the delivery tube. The flexible portion can optionally have a Durometer hardness in a range of approximately 30 to 90 on the Shore A scale, and an ultimate elongation of at least approximately 200%. The flexible portion of the delivery tubing can also or alternatively optionally include a first layer that comprises an elastomer selected from a group consisting of a silicone-based fluoroelastomer, fluoropolymer, thermoplastic elastomer, olefin elastomer, and combinations and mixtures thereof. Also or alternatively, the flexible portion of the delivery tubing can “the” are intended to include both singular and plural references unless the context clearly indicates otherwise. Thus, for example, a reference to “a tube” includes a plurality of such tubes, as well as a single tube, and any equivalents thereof.

Throughout this description, including the foregoing description of related art, any and all publicly available documents described herein, including any and all U.S. patents, are specifically incorporated by reference herein in their entirety. Descriptions of related art are not intended in any way as an admission that any of the documents described therein, including pending applications for United States patent, are prior art to the present invention. Moreover, the description herein of any disadvantages associated with the described products, methods, and/or apparatus is not intended to limit the scope of the current subject matter. Indeed, aspects of the current subject matter can include certain features of the described products, methods, and/or apparatus without suffering from their described disadvantages.

### DESCRIPTION OF DRAWINGS

The accompanying drawings, which are incorporated in and constitute a part of this specification, show certain aspects of the subject matter disclosed herein and, together with the description, help explain some of the principles associated with the disclosed embodiments. In the drawings,

FIG. 1A and FIG. 1B are schematic diagrams showing top (1A) and side views (1B) of a device configured as two part patch unit according to some embodiments of the present invention;

FIG. 2A, FIG. 2B, and FIG. 2C are detailed schematic diagrams showing a disposable part (2A), a reusable part (2B), and an assembled patch unit (2C); include a first layer of silicone rubber, polyurethane, thermoplastic elastomer or olefin elastomer. The flexible portion of the delivery tubing can optionally include an inner surface that has been treated with plasma or is fluorinated and/or can optionally include a layer of Parylene and/or an outer layer of PTFE. The flexible portion can optionally include styrene-butadiene-ethylene-styrene (SEBS). The outer layer of the flexible portion can optionally include a material that restricts molecular diffusion through the outer layer relative to the inner layer. The outer layer can also or alternatively be lubricated.

The details of one or more variations of the subject matter described herein are set forth in the accompanying drawings and the description below. Other features and advantages of the subject matter described herein will be apparent from the description and drawings, and from the claims.

For the purposes of promoting an understanding of the subject matter described herein, which can include one or more features and variations as described below, references may be made to specific implementations having discrete feature sets. The terminology used herein is for the purpose of describing particular implementations only, and is not intended to limit the scope of either the disclosed subject matter or of the particular inventions that are claimed below. For example, references to the delivery of insulin and/or the treatment of diabetes are intended to serve as illustrative examples of broader inventive concepts. Use of other therapeutic fluids, either in addition to or in place of insulin, as well as materials and apparatuses compatible with the same, are within the contemplated scope of the current subject matter. Additionally, as used throughout this disclosure, the singular articles “a,” “an,” and

FIG. 3 is a schematic diagram showing a delivery tube and peristaltic pump rollers;

FIG. 4 is a schematic diagram showing a cross-sectional view of a composite tube;

FIG. 5A and FIG. 5B show chemical structure diagrams of two polysiloxane polymers, one non-fluorinated (5A) and the other fluorinated (5B), that could be sued with the current subject matter;

FIG. 6A, FIG. 6B, and FIG. 6C are schematic diagrams of a tube provided with one or more unidirectional valves;

FIG. 7A, FIG. 7B, and FIG. 7C are schematic diagrams showing a rotating wheel and convex rollers; and

FIG. 8A, FIG. 8B, and FIG. 8C are schematic diagrams showing cross section of delivery tube pressed between a concave stator and convex roller; and

FIG. 9 is a process flow chart showing a method for delivering therapeutic fluid to a patient via a delivery tubing.

### DETAILED DESCRIPTION

The chemical and/or physical characteristics of the delivery tube used in a fluid delivery device can have a substantial effect on the functionality and usefulness of the device. In general, the clinical efficacy of a delivered insulin formulation can be highly dependent on its chemical stability. Rapid acting insulin analogs, such as for example Humalog, Novolog, lispro, aspart, and glulisine, as well as other formulations of insulin and/or other therapeutic compounds, can include sensitive monomers carried within a preservative, such as for example m-cresol, phenol, or the like. To ensure that insulin reaches the body intact and with its intended potency, the chemical properties of the monomer and of the preservative ideally remain unchanged during storage of the formulation in a delivery device reservoir and during displacement from the reservoir to the patient's body through a delivery tube. As such, compatibility of the delivery tube with insulin is of utmost importance. Unfortunately the most commonly used materials for manufacturing flexible tubing for peristaltic pumps such as silicone rubber and polyvinyl chloride (Tygon®) may not be as compatible with insulin formulations as would be desirable.

One or more insulin compatibility issues can arise with currently available delivery tubing, including but not necessarily limited to chemical (covalent) aggregation and precipitation, physical (non-covalent) aggregation (fibrillation), insulin absorption on the inner walls of the tubing, absorption or diffusion through tubing walls by preservatives in the insulin solution and/or by atmospheric contaminants such as carbon dioxide, shedding of particulate matter from the inner tubing walls into the insulin solution, and excessive water and/or alcohol permeation out of a delivered insulin solution. Some of the factors that may be associated with each of these issues are briefly summarized below.

Insulin tends to precipitate at elevated temperature and/or low pH. This reaction can result in reduction of biological activity by the insulin. Precipitated insulin can aggregate to form particles that might lead to catheter or tubing occlusion or clogging, thereby slowing or possibly completely interrupting delivery of insulin to a subject's body. Insulin solution pH can be lowered due to diffusion of CO₂ through plastic components of a delivery system. Such diffusion can lead to the formation of carbonic acid in the presence of an aqueous solution. Rapid acting insulin generally precipitates at a pH of about 3.5-6 (Diabetes Technology & Therapeutics 2007; 9 (1): 26-35). Continuous insulin infusion devices may also expose insulin to mechanical conditions such as agitation, sheer stress, and contact with rough and hydrophobic surfaces for extended periods of time. These conditions can increase non-covalent insulin precipitation. In the formation of insulin fibrils, partially unfolded insulin molecules may interact with each other to form linear aggregates (J. Pharm. Sci. 1997; 86(5): 517-25).

Insulin also tends to adsorb on inner surfaces of some types of tubing material. For example, insulin generally binds to PVC tubing until all binding sites are saturated. This interaction can lower the insulin concentration in a formulation flowing through such a tube, thereby decrease the delivered dose. The concentrations of preservatives, such as for example m-cresol or phenol, in insulin solutions can also be reduced due to absorption into tubing materials and/or diffusion through tubing materials. This can be a major concern with many currently available types of insulin pump tubing. (Chantelau et al, 1987, Sartorius, 1988; Melberg et al., 1988.)

Some types of currently available tubing can have a tendency to shed particulate matter into solution. Such spalling of tubing material can generate contaminating particulates in the delivered insulin solution (Pharmaceutical Development and Technology, Volume 7, Number 3/2002 pp. 317-323). Excessive water and/or alcohol evaporation from a delivered insulin formulation, such as for example due to permeation of water and alcoholic preservatives through the walls of all-silicone or other types of tubing, can also occur. Permeability of a piece of tubing is generally inversely related to wall thickness. In some examples, the small size tubing used in currently available miniature insulin pumps can allow the water content of a solutioned contained therein to be depleted within about 8-18 hours. (Diabetes Care 3(2): 322-331).

Tubes that are compatible with insulin and which are used to deliver insulin to the body can include an inner layer of polyolefin, such as for example polyethylene, polypropylene, or the like; a middle bonding layer; and an outer layer of PVC. Polyolefin plastics have fewer binding sites, are more resistant to carbon dioxide penetration, and leach fewer materials into solution than such plastic like PVC. The outer layer provides kink resistance and strength better than can be achieved with the use of polyolefin alone. U.S. Pat. Nos. 5,702,372 1997 and 6,302,990, both of which are incorporated herein by reference in their entireties, each describe double-layer (“jacket”) tubing for insulin-contact applications. The catheter inner layer of these tubes is compatible with the delivered fluid and the outer layer is a flexible elastomer. U.S. Pat. No. 6,530,912, also incorporated herein by reference in its entirety, discloses and claims a system for connecting a reservoir of a delicate therapeutic formulation with a lumen. The system can include an inner lumen lined with a “therapeutic compatible lining” that can shield the therapeutic fluid from contacting materials that can diminish the integrity of the therapeutic fluid. These tubes, however, lack the properties required for delivery of insulin via a delivery tube coupled to a peristaltic pump because they are generally stiff and not elastic.

Various implementations of the currently disclosed subject matter can provide infusion devices composed of a miniature skin adherable infusion patch unit (also referred to herein as a “dispensing patch”) that can in some examples be remotely controlled by a remote control unit. The dispensing patch can be discreet and free of inconvenient tubing and can dispense insulin or some other therapeutic compound or formulation via a delivery tube that is coupled to a peristaltic pump. The dispensing patch can in some examples include a disposable part and a reusable part. The reusable part can optionally contain relatively expensive components of the dispensing patch (such as for example a peristaltic pump wheel, driving mechanism and electronics) and the disposable part can optionally contain relatively less expensive components (such as for example a delivery tube, reservoir, and the like). In this manner, a device can have relatively low ongoing operating costs for a user while being highly profitable for manufacturers and payers.

Dispensing patch units consistent with various aspects of the current subject matter can include a peristaltic pumping mechanism (peristaltic pump). The peristaltic pump can optionally include a rotatable structure such as a wheel that includes one or more rollers, a stator and a delivery tube. The wheel and rollers can in some variations be located within a reusable part of the dispensing patch and a delivery tube and stator can be located within a disposable part. After a disposable and reusable part are properly paired, the wheel can be rotated and the rollers can squeeze the tube against the stator. Fluid delivery can thereby be maintained in the direction of rotation of the wheel.

In some implementations, only the segment or portion of the delivery tube that comes in contact with the rollers of a peristaltic pump need be elastic. Other portions of the tube may be rigid. The entire length of the tube can advantageously be biocompatible with the delivered fluid.

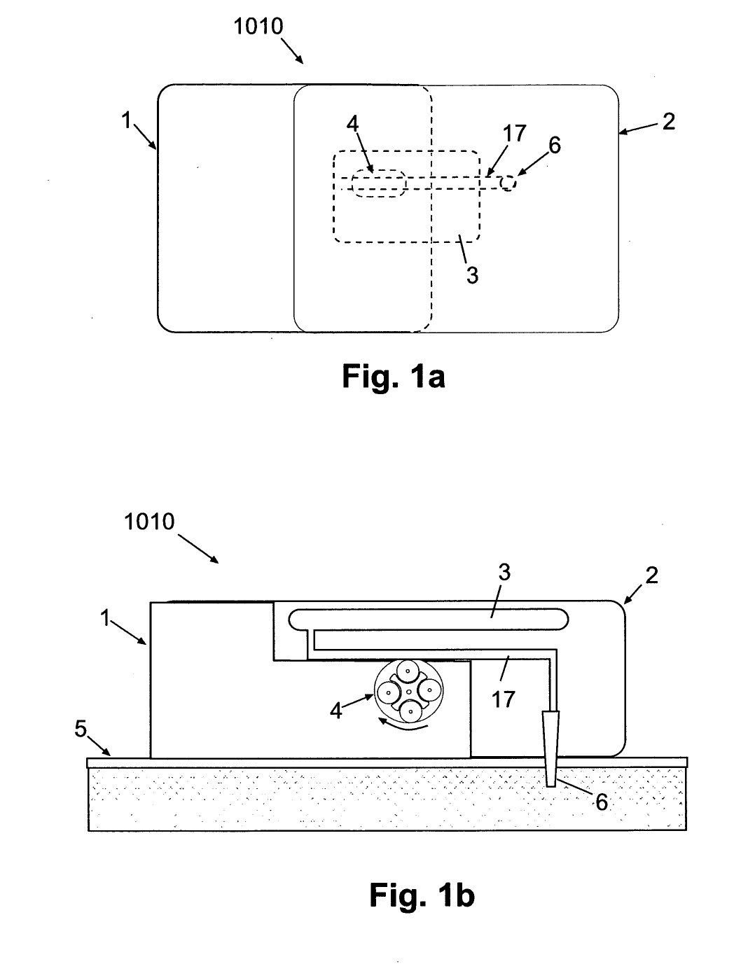

FIG. 1A and FIG. 1B show preferred embodiment of a two-part dispensing patch unit 1010. FIG. 1A shows a top view of the dispensing patch unit 1010 composed of a reusable part 1 and a disposable part 2. FIG. 1B shows a side view of the dispensing patch unit 1010. The disposable part 2 includes a reservoir 3, a delivery tube 17, and a cannula 6 that can be insertable into a user's skin 5. The reusable part 1 can include a pump mechanism 4 that includes rotating wheel and rollers, a driving mechanism, and one or more electronic components. In an alternative variation, a cradle unit can be provided as described in currently pending and co-owned applications for U.S. Patent No. 60/876,679 and Ser. No. 12/116,546, which are incorporated herein by reference in their entireties. The cradle of such a device can be adhered to a user's skin 5, thereby allowing connection and disconnection of the dispensing patch unit 1010. The cannula 6 can be automatically or manually inserted into a patient's body through an opening of the cradle unit.

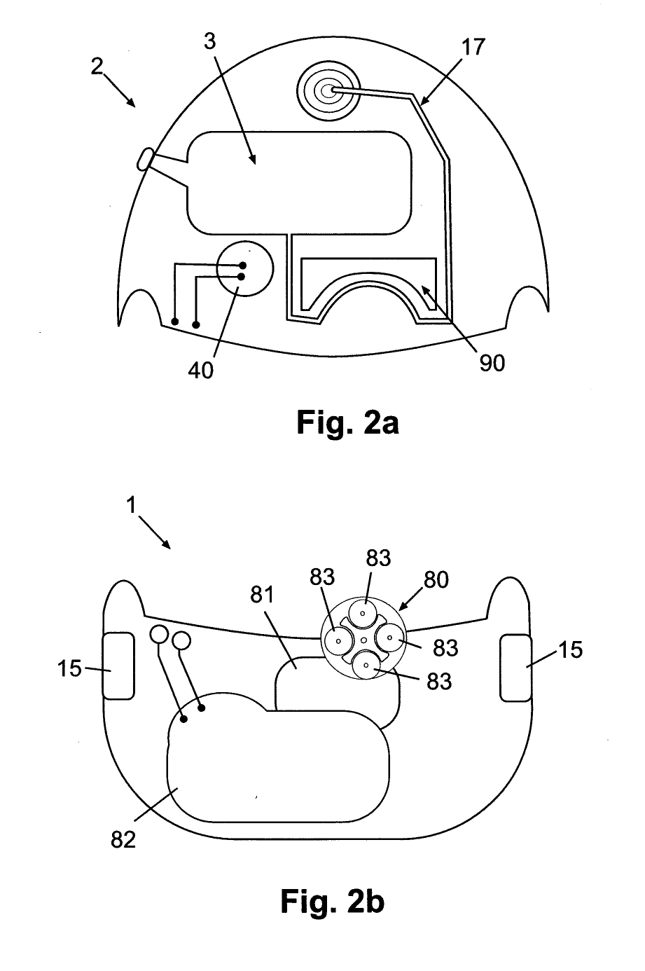

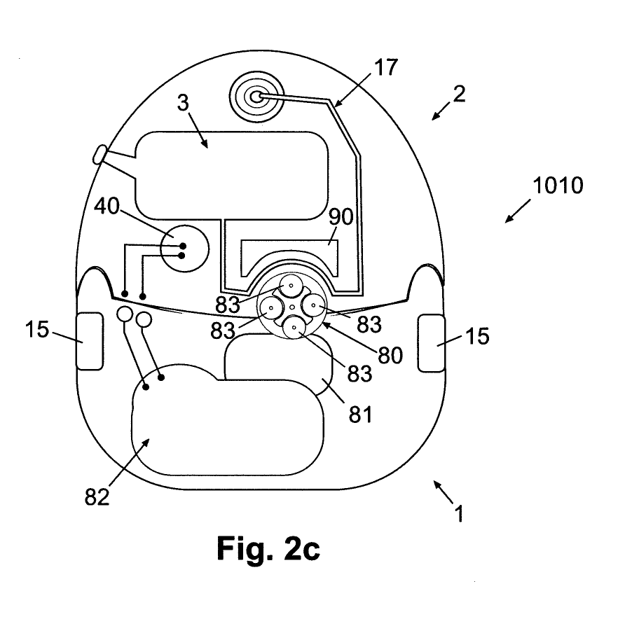

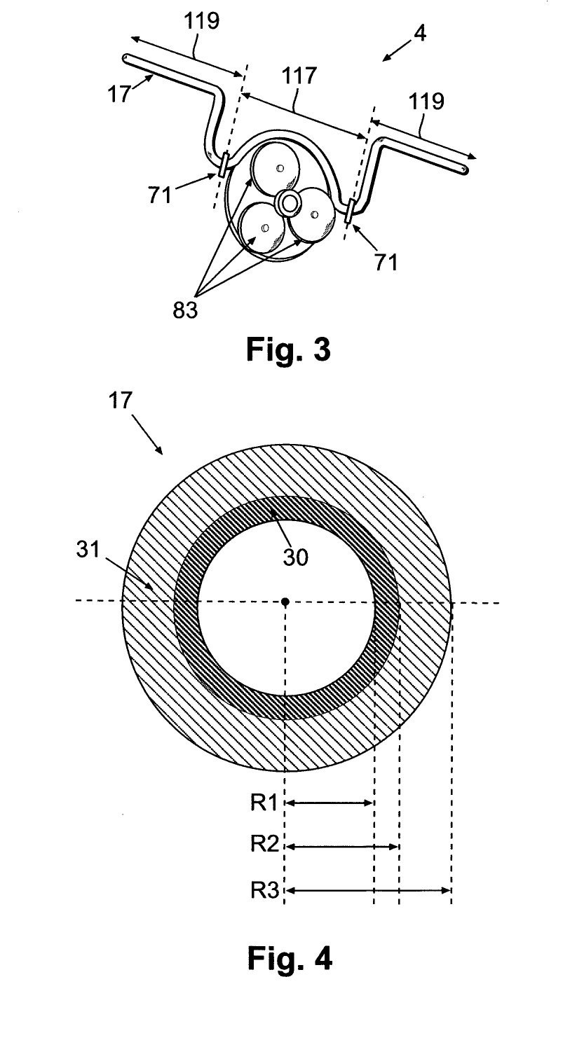

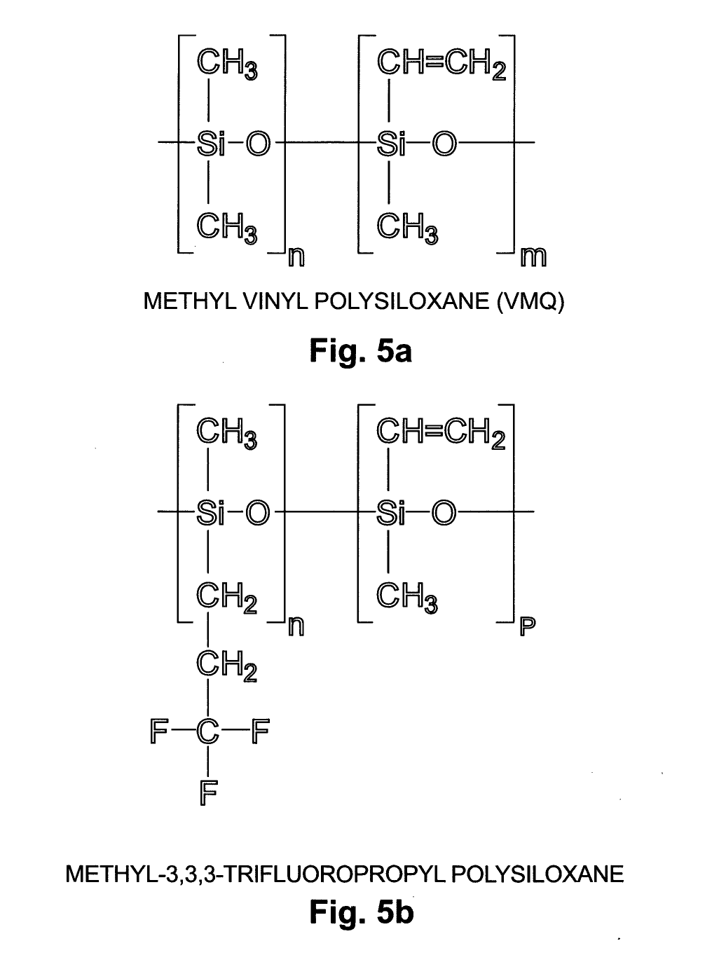

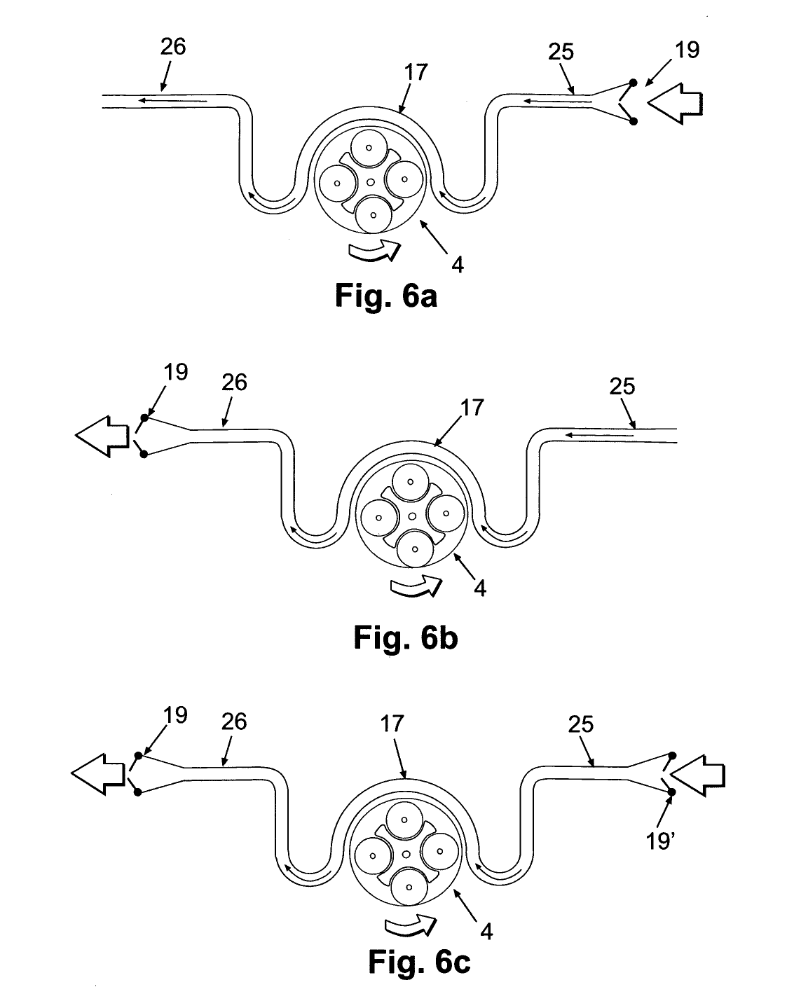

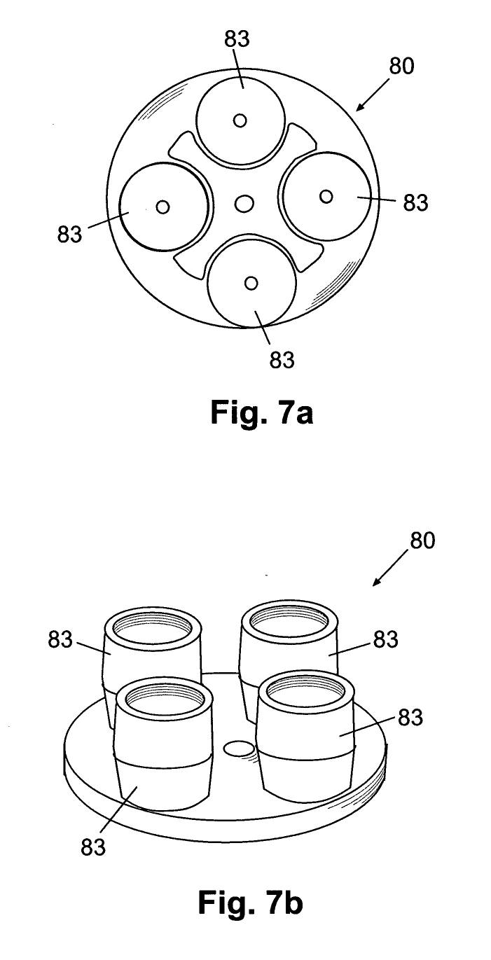

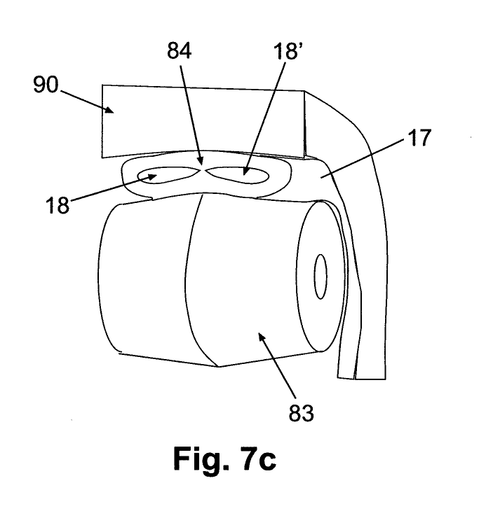

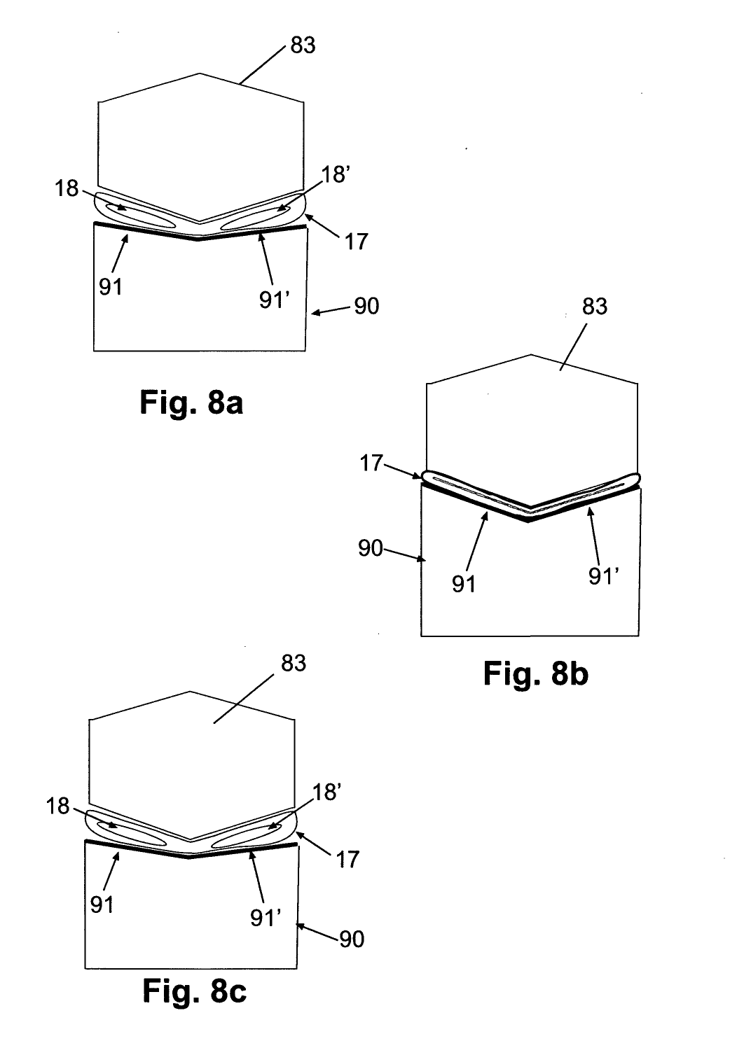

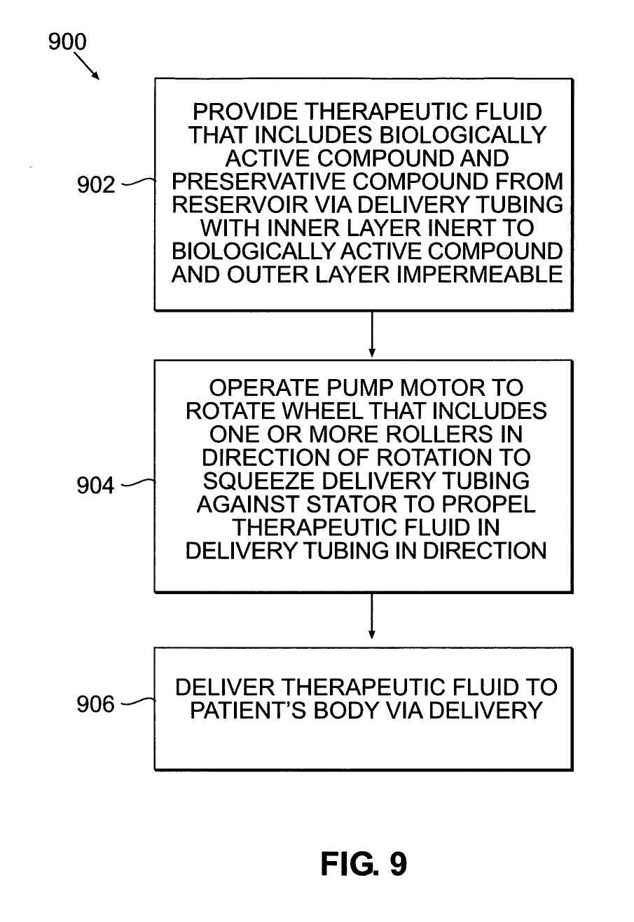

FIG. 2A, FIG. 2B and FIG. 2C show in detail an example of a two-part dispensing patch unit 1010. FIG. 2A shows a disposable part 2 that includes a delivery tube 17, a stator 90, an infusion bag 3 that can contain insulin or another therapeutic formulation, and a power supply 40 that can be, for example a battery, a fuel cell, or any other device or feature capable or providing power. FIG. 2B shows a reusable part 1 that includes a peristaltic pump rotating wheel 80 with rollers 83, a driving mechanism 81, one or more electronic components 82, and one or more buttons 15 that can optionally be included to enable manual control of delivery of insulin or the therapeutic formulation, for example by commanding the delivery of a bolus. FIG. 2C shows the reusable part 1 and disposable part 2 after they have been connected. After connection of both parts, the rollers 83 of the rotating wheel 80 of the reusable part 1 can squeeze the delivery tube 17 of the disposable part 2 against the stator 90.

FIG. 3 shows a delivery tube 17 and rollers 83 of a peristaltic pumping mechanism 4. In one example, at least a first resilient portion 117 of the tube which comes in contact with the rollers 83 can be configured to ensure mechanical compatibility with the peristaltic pumping mechanism. This mechanical configuration can include, for example, sufficient resiliency and/or side wall thickness to enable use within the peristaltic pumping mechanism. Other portions of the tube 119 can advantageously be made compatible with insulin or other therapeutic formulations or preparations and can have mechanical properties that differ from the resilient portion 117 of the tube. For example, because the other portions 119 of the tube need not interact with the rollers 83 and stators of the peristaltic pump, they can optionally be rigid or semirigid. The resilient portion 117 of the tube 17 can be connected to the other portions 119 by any suitable connector, connection device, or other connections means, including but not limited to metal or plastic tubular connectors. These connectors are schematically shown in FIG. 3 and designated by numeral 71.

A delivery tube 17 can in some implementations be compatible with commercial short acting insulin analog preparations. The delivery tube can also or alternatively be configured with suitable physical properties to comply with the peristaltic pumping mechanism. The tube can also have one or more properties that facilitate maintaining physical and chemical properties of insulin preparation for a desired time period (such as for example at least three consecutive days). Various aspects of the device and delivery tubing can be designed to maintain insulin stability, to avoid fibrillation, and/or to reduce preservative evaporation. Insulin stability factors can include, but are not limited to insulin potency, preservative concentration, A21-desamido acid, high molecular weight components, other related substances, pH level of the formulation, and the like.

FIG. 4 shows a cross sectional view of an example of a composite delivery tube 17 of the invention. The delivery tube 17 can include at least two layers: an inner layer 30 that is inert or at least chemically resistant to insulin and/or other therapeutic preparations or formulations, and an outer layer 31 that is made of an elastomer that is resilient. The two layers can be joined by adhesion, crosslinking, or other methods. An additional adhesive layer (not shown) can also be included between the inner layer 30 and outer layer 31.

According to some implementations, the inner layer 30 can include a biocompatible fluorosilicone and the outer layer 31 can include a material, such as for example parylene, that prevents or at least reduces permeation of water and alcoholic preservatives from the insulin formulation through the walls of a fluorosilicone or other type of delivery tube. The parylene coating can advantageously have a uniform or approximately uniform thickness and be substantially free of pores. Such a material can in some implementations be achieved by chemical vapor deposition polymerization. One possible advantage of a chemical vapor deposition polymerization process is that the coating forms from a gaseous monomer without an intermediate liquid stage.

FIG. 5A shows the chemical structure of a non-fluorinated silicone that can be used in elastomers. Fluorosilicone elastomers resistance to m-cresol preservatives, coupled with their favorable elasticity, low compression set, and reduced gas permeability relative to non-fluorinated silicone elastomers, make these materials attractive candidates for small-diameter peristaltic tubing for use with aqueous insulin formulations. Fluorosilicone is a siloxane polymer (silicon-oxygen bonds in the polymer backbone of silicone polymers) that includes units of methyl-3,3,3-trifluoropropyl polysiloxane. When used to make elastomers, the fluorosilicone polymer structure includes both methyl-trifluoropropyl siloxane units and methyl-vinyl siloxane units and is referred to as fluorovinylmethyl silicone rubber, or FVMQ.

FIG. 5B shows the chemical structure of a fluorosilicone polymer that can be used in elastomers (chemical name trifluoropropylmethyl siloxane) disclosed herein. The (—CF₃) group has a polar nature which imparts the fluorosilicone polymers with superior resistance to many chemicals including insulin. Trifluoropropyl groups situated along the chain change the solubility parameter of the polymer from 7.5 cal^{1/2}·cm^{-3/2} to 9.5 cal^{1/2}·cm^{-3/2}. (Chimie Nouvelle, vol. 8 (30), 847 (1990) by A. Colas from Dow Corning Corporation). In general, the solubility parameter has a somewhat linear relationship with surface energy. The higher the surface energy, the less hydrophobic the polymer. Thus, the overall fluorosilicone structure is less hydrophobic than non-fluorinated silicone.

Fluorosilicone can also prevent pH decreases that might result from diffusion of CO₂ gas from the surrounding atmosphere into the formulation being conveyed in the delivery tube. This is a result of a fourfold to fivefold lower gas permeability of the fluorinated-silicone in comparison with non-fluorinated silicone (ref: Dow Corning web site). When insulin is kept in standard containers its pH level is about 7-8. It can be desirable to retain this pH level since the isoelectric precipitation point for insulin occurs at approximately a pH of 6. Below this pH level, insulin aggregations are more likely to occur and thereby reduce the functionality and/or potency of the insulin formulation delivered to the patient.

A delivery tube consistent with the current subject matter can include an inner layer of fluorosilicone and an outer layer. The outer layer can be configured or formulated to reduce or otherwise impede evaporation of water, alcoholic preservatives such as phenol and m-cresol, and/or other constituents of the insulin or other therapeutic formulation. As such, the outer layer individually and the delivery tubing as a whole can be substantially impermeable to both liquid and vapor phases of one or more of the preservative compounds used in the formulation or preparation. Likewise, the outer layer individually and the delivery tubing as a whole can be substantially impermeable to both liquid and vapor phases of water. The outer layer can in some implementations be made of an elastomeric material such as rubber (i.e. butyl, chlorobutyl, etc.), Viton®, Sanipure®, etc or very thin (1-10 micron) rigid layer such as PTFE (Teflon), Parylene, etc. In another optional variation, a delivery tube can include an oily outer layer such as a lubricant oil, that can in one example include petroleum jelly or the like.

The delivery tubing can also and/or alternatively include a layer of an elastomer, such as for example one or more of a silicone-based fluoroelastomer, fluoropolymer, thermoplastic elastomer, olefin elastomer and combinations and mixtures thereof. Delivery tubing can also and/or alternatively include a layer of silicone rubber, polyurethane, thermoplastic elastomer or olefin elastomer. The inner surface of the delivery tubing can optionally be treated with plasma or be fluorinated to reduce chemical interactions with components of the insulin or other therapeutic formulation being delivered. Alternatively or in addition, an outer layer can be provided that includes a material which prevents diffusion of molecules through the outer layer. In some implementations, delivery tubing can include a fluorosilicone rubber inner layer that is coated with an anti-evaporating layer. An additional adhesive layer can optionally be incorporated between one or more layers of the delivery tubing walls.

The inner layer of the tube can in some examples be of low density polyethylene, and the outer layer may be manufactured from silicon, polyurethane, butyl rubber, styrene-butadiene-ethylene-styrene (SEBS) or ethylene-vinyl-acetate (EVA) elastomer. Polyethylene is less likely to promote insulin aggregation than alternative materials such as PVC. The thickness of the inner layer can in some examples be about several micrometers (e.g. 15μ). A polymer tubular member can optionally be co-axially disposed over such an inner layer. The co-axially disposed polymer imparts the tube with the required elasticity. The co-axially disposed polymer can, in some examples be PTFE. Another possible outer layer for a composite delivery tube according to the current subject matter can comprise a metallic dispersant. For example, a composite tube can include a fluorosilicone inner layer covered with an outer dispersion of gold.

Additional tubes according to the current subject matter can include a layer (in some examples a single layer) of modified silicone or polyurethane. The modification comprises the addition of functional groups (“surface active end groups”) to the polymer molecule. According to one such embodiment, the tube comprises a plasma treated inner surface and that is suitable for peristaltic pump use. Plasma treatment can increase surface crosslinking and thereby reduce adsorption of various molecules. An alternative method for reducing adsorption of various molecules can be fluorination of the inner surface.

A layer of fluorosilicone (in some examples a single layer) can be used on the inner surface of a delivery tube 17. Solubility of many chemicals can be decreased by the addition of electronegative side-groups to the polymer chain. Fluorine can provide this electronegative character. Flexible tubing made from fluorinated polymers performed well in terms of low sorption of pharmaceutical preservatives (Pharmaceutical Development and Technology, 7(1), 49-58, 2002). In addition, insulin fibrillation is decreased in fluorosilicone tubing.

A layer (in some examples a single letter) of non-silicone-based fluoroelastomers, such as for example Chem-Sure®-Gore, expanded PTFE, and viton, can also be used on the inner surface of a delivery tube 17. A single layer or multiple layers of a thermoplastic elastomer, such as for example Santoprene®-Advanced Elastomer Systems, Sani-Pure®-St. Gobain, can also be used on an inner surface of a delivery tube 17. Thermoplastic elastomers (TPE's) are block copolymers with alternating “soft” and “hard” segments. The hard segments can act as rigid anchors upon deformation, allowing the material to return to its original dimensions upon release of applied stress. One or more layers of an olefin elastomer, such as for example Vistamaxx, can also be used as the inner surface. In this an other implementations, the flexible portion of the delivery tubing can optionally have a Durometer hardness in a range of approximately 30 to 90 on the Shore A scale. In this and other implementations, the flexible portion of the delivery tubing can also optionally have an ultimate elongation of at least approximately 200%.

According to some embodiments of the present invention, the peristaltic pump may include convex rollers and concave stator design that promotes gentle pumping. The described configuration enables the rollers to gradually squeeze the tube. The tube cross section is squeezed first in its mid portion followed by squeezing at the edges. Such a configuration may reduce the force induced insulin physical fibrillation.

Physical impact forces applied in the process of moving a fluid can result in aggregation, fibrillation, and/or other mechanically induced, undesirable changes to an insulin or other therapeutic formulation. Insulin fibrillation and/or aggregation can be induced by a peristaltic pumping mechanism which causes repetitive pressure on insulin molecules and consequently leads to spatial change and aggregation. Some implementations of the current subject matter therefore can provide a more gradual change of the spatial conformation of the insulin molecules during the action of a peristaltic pump mechanism and thereby reduce the impact forces exerted by the rollers 83 and the stator 90 on fluid contained in the delivery tube 17. Thus, the occurrence of the undesired effects can be decreased while maintaining desired functionality of insulin preparation.

Examples of structures and methods that can reduce the pressure and physical forces applied to insulin and other therapeutic formulations are shown in FIG. 6, FIG. 7, and FIG. 8. FIG. 6A, FIG. 6B, and FIG. 6C show examples of how one or more unidirectional valves can be incorporated into the fluid delivery path to prevent backflow of delivered fluid. Backflow can happen when one roller of peristaltic wheel leaves the stator curvature before adjacent roller enters. Prevention of backflow allows a reduction of the counter-force applied by the peristaltic pump wheel and rollers and the stator on the delivery tube and consequently can reduce the incidence of induced insulin physical fibrillation. Moreover, prevention of backflow allows for more accurate dosing, both of the basal insulin dosing and of manually or automatically delivered boluses. In various implementations, unidirectional valves can be placed at the tube outlet or at both tube inlet and outlet.

In FIG. 6A the valve 19 is located at an inlet 25 to the delivery tube 17. In FIG. 6B the valve 19 is located at the outlet 26 to the delivery tube. In FIG. 6C a first valve 19 is located at the delivery tube outlet 26 and a second valve 19′ is located at the delivery tube inlet 25. One or more unidirectional valves 19 can prevent backward flow of insulin or other therapeutic formulation that may otherwise occur when one of the rotating wheel rollers 83 leaves the stator before the adjacent roller enters.

FIGS. 7A, 7B and 7C show various views of convex shaped rollers 83 that are mounted on a rotating structure, such as for example a wheel 80. The convex shape of the rollers 83 as shown in these diagrams can enable gradual squeezing of the delivery tube 17 against the stator 90, thereby reducing undesired effects, such as for example aggregation and/or fibrillation of insulin molecules. Such a mechanism can provide the benefit of improved retention of insulin preparation functionality and efficacy. FIG. 7A is a transverse profile of the rotating wheel 80 with four rollers 83. FIG. 7B shows the convex shaped rollers 83, which in this example are approximately barrel-shaped, seating on the rotating wheel 80. A rotating wheel can mount any number of rollers. In some implementations, three or four rollers can optionally and advantageously be used.

FIG. 7C shows an example of how the convex shaped rollers 83 press the delivery tube 17 against the stator 90. The stator 90 can be designed so that when initial pressing occurs, only the central region 84 of the convex shaped roller 83 contacts the stator, thus squeezing the tube by the lateral portions of the roller in such a manner that two lumens 18 and 18′ are created within the delivery tube 17. The lumens allow passage of insulin molecules that have been squeezed to the sides. As the rotation of the wheel continues, pressing of the rollers proceeds, such that the rollers 83 press the insulin within the lumens 18 and 18′ forward.

FIG. 8A, FIG. 8B, and FIG. 8C show three cross sectional views of a roller 83 at a first (FIG. 8A), middle (FIG. 8B) and last (FIG. 8C) portions of the stator 90 in the direction of rotating wheel motion. The lumens 18 and 18′ can be formed when the stator angles of inclination 91 are changed.

Another optional feature that can be included in one or more of the above-discussed implementations is fin that is attached to or otherwise formed as part of the outer surface of a delivery tube for a peristaltic pump. The fin can be caged or otherwise secured in a slit formed between two parts of the stator, thus securing the delivery tube on the stator. This feature can prevent or limit the negative impact of stretching of the delivery tube during the operation of the peristaltic pump, thereby allowing smoother pumping motion, avoidance of rotor jamming by a pulled-out delivery tube, and reduction of friction of the delivery tube against the stator. The fin can optionally extend along the entire length of the delivery tube or only along the part of the delivery tube that comes in contact with the rollers of the peristaltic pump.

In a further implementation of the current subject matter, methods are provided for dosing a patient with a therapeutic fluid provided in a reservoir. FIG. 9 shows a flow chart that summarizes an example of such a method. At 902, a therapeutic fluid can be provided from a fluid reservoir via a delivery tubing. The fluid reservoir can be as described above or other reservoirs that are adaptable to provide fluid via an exit tubing. The therapeutic fluid can include at least one biologically active compound and at least one preservative compound. The delivery tubing has a flexible portion that can include an inner layer that is substantially inert to reactions with the at least one biologically active compound and an outer layer that is substantially impermeable to gas and liquid phases of at least the preservative compound and/or water. At 904, a pump motor is operated to rotate a wheel in a direction of rotation. The wheel can include one or more rollers and be disposed such that the one or more rollers squeezes the flexible portion of the delivery tubing against a stator to propel the therapeutic fluid in the delivery tubing in the direction of rotation as the wheel rotates. At 906 the therapeutic fluid is delivered to a patient's body via the delivery tubing, which can, for example, be connected to a cannula at the end of the delivery tubing opposite the reservoir. Such a cannula can be disposed to penetrate at least the outer layer of a patient's skin to deliver the therapeutic fluid to the subcutaneous compartment.

The subject matter described herein may be embodied in systems, apparatus, methods, and/or articles depending on the desired configuration. The implementations set forth in the foregoing description do not represent all implementations consistent with the subject matter described herein. Rather, they are merely some examples consistent with aspects related to the described subject matter. Wherever possible, the same reference numbers will be used throughout the drawings to refer to the same or like parts. Although a few variations have been described in detail above, other modifications or additions are possible. In particular, further features and/or variations may be provided in addition to those set forth herein. For example, the implementations described above may be directed to various combinations and subcombinations of the disclosed features and/or combinations and subcombinations of several further features disclosed above. In addition, the logic flow depicted in the accompanying figures and/or described herein do not require the particular order shown, or sequential order, to achieve desirable results. Other embodiments may be within the scope of the following claims.

1.-23. (canceled)

24. A portable ambulatory, therapeutic fluid dispensing device comprising:
a reservoir retaining a therapeutic fluid, the therapeutic fluid comprising at least one biologically active compound and at least one preservative compound, wherein the at least one biologically active compound comprises insulin;
a peristaltic pump for propelling the therapeutic fluid from the reservoir to a body of a patient; and
a delivery tube including a flexible portion, wherein the flexible portion comprises an inner layer substantially inert to reactions with the at least one biologically active compound and an outer layer that is substantially impermeable to both liquid and vapor phases of at least the preservative compound and/or water.

25. The device of claim 24, wherein the peristaltic pump comprises:
a pump motor;
a stator; and
a wheel rotatable in a first direction by the pump motor, the wheel comprising one or more rollers, the wheel being configured such that the one or more rollers squeezes the flexible portion of the delivery tube against the stator to propel fluid in the delivery tube in the first direction as the wheel rotates.

26. The device of claim 24, wherein the delivery tube further comprises a rigid portion which is substantially impermeable to at least one of the therapeutic fluid components and/or to CO₂ than the flexible portions of the delivery tube.

27. The device of claim 24, wherein the flexible portion has a Durometer hardness in a range of approximately about 30 to about 90 on the Shore A scale, and an ultimate elongation of at least approximately 200 percent.

28. The device of claim 24, wherein the flexible portion of the delivery tube comprises a first layer comprising an elastomer, the elastomer being selected from a group consisting of a silicone-based fluoroelastomer, fluoropolymer, thermoplastic elastomer, olefin elastomer, and combinations and mixtures thereof.

29. The device of claim 24, wherein the flexible portion of the delivery tube comprises a first layer including one or more of silicone rubber, polyurethane, thermoplastic elastomer and olefin elastomer.

30. The device of claim 24, wherein the flexible portion of the delivery tube comprises an inner surface having a plasma treatment or is fluorinated.

31. The device of claim 1, wherein the inner layer is substantially more inert to the biologically active compound than the outer layer.

32. The device of claim 31, wherein the inner layer includes fluorosilicone.

33. The device of claim 24, wherein the flexible portion comprises a layer of Parylene.

34. The device of claim 24, wherein the flexible portion comprises styrene-butadiene-ethylene-styrene (SEBS).

35. The device of claim 24, wherein the outer layer of the flexible portion restricts molecular diffusion through the outer layer relative to the inner layer.

36. The device of claim 24, wherein the flexible portion comprises an outer layer of PTFE.

37. The device of claim 24, wherein the outer layer is lubricated.

38. The device of claim 25, wherein the rollers include a convex cross section in a plane substantially parallel to the axis of rotation of the wheel.

39. The device of claim 38, wherein the stator comprises a contact surface that is substantially parallel to the axis of rotation and wherein the flexible portion of the delivery tube is compressed between the convex cross section of the roller and the stator to substantially form two parallel lumens within the delivery tube.

40. The device of claim 39, wherein the stator comprises a concave cross section.

41. The device of claim 24, further comprising a valve to allow unidirectional propulsion of the therapeutic fluid in the delivery tube.

42. The device of claim 24, wherein the at least one preservative compound comprises one of: phenol, phenol derivative, and cresol.
